(19) United States
(12) Patent Application Publication
(10) Pub. No.: US 2003/0059850 A1
(43) Pub. Date: Mar. 27, 2003

(54) Fluorescence proximity assay

(75) Inventor: David Mark Evans, N. Potomac, MD (US)

Correspondence Address: DARBY & DARBY P.C., Post Office Box 5257, New York, NY, 10150-5257, US

(73) Assignee: Psychiatric Genomics, Inc.

(21) Appl. No.: 10/256,091

(22) Filed: Sep. 26, 2002

Related U.S. Application Data
(60) Provisional application No. 60/325,269, filed on Sep. 26, 2001.

(51) Int. Cl. C12Q 1/68; G01N 33/53; G01N 33/551

(52) U.S. Cl. 435/7.1; 436/524; 435/6

(57) ABSTRACT

The present invention provides binding assays, referred to here as fluorescence proximity assays or FPA. The inventions detect binding of target molecules in a sample to a molecular probe or probes that specifically bind or hybridize to those molecules. In particular, the molecular probes are immobilized to a bead or particle, such as colloidal gold, the reflects fluorescent energy from a fluorophore. The derivatized beads are contacted to a sample of fluorescently labeled target molecules, and binding of the target is indicated by an increase in the fluorescent signal. Kits are also provided that contain materials and reagents to performing a fluorescence proximity assay.

### 1. PRIORITY INFORMATION

Priority is claimed under 35 U.S.C. §119(e) to U.S. provisional patent application Serial No. 60/325,269 filed on Sep. 26, 2001, which is incorporated herein by reference in its entirety.

### 2. FIELD OF THE INVENTION

The present invention relates to binding assays for detecting the presence of particular molecules in a sample, such as particular polypeptides or particular nucleic acid sequences. In preferred embodiments, the invention relates to homogenous binding assays that use molecular probes attached to a particle or bead (e.g., colloidal gold), as opposed to probes that are immobilized on a membrane or other solid surface.

### 3. BACKGROUND OF THE INVENTION

High throughput specific binding assays provide an important tool in fields such as molecular biology and medical diagnostics. For example, nucleic acid molecules are typically detected in biological samples by hybridization to complementary nucleic acid probes. Generally, the probes are immobilized on a surface such as a nitrocellulose filter (e.g., for Southern blot assays) or the bottom of a microtiter plate (e.g., for microarrays). Similarly, Western blotting assays detect polypeptide molecules by binding to an antibody that is immobilized on a solid surface.

A significant problem with the implementation of such assays is the need to wash the sample and remove unbound ligand molecules. This adds additional, often time consuming steps to the assays, complicating the procedure and reducing throughput. Moreover, it is often desirable to perform specific binding assays with soluble materials or living cells, which are not amenable to a washing step. Some alternative assay methods are known that do not require a wash step. However, these assays also suffer from technical drawbacks that may outweigh the advantage of eliminating a wash step.

For example, confocal microscopy methods are known that rely on the confocal microscope's discrimination of a very small depth. See, .e.g, in Moore et al., J. Biomol. Screening 1999, 4(6):335-354. In such methods, measurements are made from the underside of a surface to which fluorescently labeled target molecules are attracted, e.g., by the attachment or immobilization of a target specific probe or cells. Such assays are limited, however, by the optical clarity of the surface through which measurements are made. In addition, the procedure requires use of a flat surface and, consequently, a small surface area to volume ratio for the immobilization surface. This limitation results in a diminished signal per unit area. In addition, confocal imaging systems are able to interrogate only a small area of the immobilization surface at a time. It is therefore necessary to scan as much of the immobilization surface as possible, making the assay time consuming and reducing throughput for multiple samples. Perhaps more significantly, the confocal imaging systems required to implement this type of assay are expensive and complicated.

Homogenous assay methods are also known, in which the probe molecules are not bound to any substrate and bind target molecules in a homogenous phase (for example, in a liquid solution or in a colloidal suspension of particles). The scintillation proximity assay (SPA) is one common example of such a homogenous assay. See, e.g., U.S. Pat. No. 5,665,562. In such an assay, target specific probe molecules are attached or immobilized on the surface of a bead that contains a scintillant buried within it. Binding of a radio labeled target molecule to a specific probe therefore brings a radio isotope in close proximity to the bead so that there is a transfer of energy between the radio isotope and the scintillant, causing the emission of light which is then detected. These assays, however, are limited to the use of radio isotope labels, which require special handling procedures to protect users and the environment from radioactivity.

Still other assays have been described that use Fluorescence Resonance Energy Transfer (FRET) to detect nucleic acid sequences in a homogenous assay. See, for example, U.S. Pat. Nos. 5,573,906 and 6,090,552. Such assays typically rely on the formation of nucleic acid “hairpin” structures in self-complementary regions of a polynucleotide probe, to bring a fluorescence emitter and quencher moiety in close proximity to each other. Such assays, however, are complicated by the requirement for two additional labels, and typically have only limited applications.

### 4. SUMMARY OF THE INVENTION

The present invention overcomes problems in the prior art and provides novel binding assays (referred to here as “fluorescent proximity assays” or FPAs) that are flexible, simple and easy to use. These assays are based, at least in part, on the discovery that when a fluorescent molecule or label is brought within close proximity of a gold or other metallic bead, the fluorescent signal intensity is not quenched as might be expected (see, for example, Duhachek et al., Anal Chem. 2000, 72:3709-3716; Enderlein, Biophys J. 2000, 78:2151-2158; Ruppin, J. Chem. Phys. 1982, 76:1681-1684; and Pineda et al., J. Chem. Phys. 1985, 83:5330-5337). Rather, the close proximity of the metallic bead to the fluorescent moiety actually enhances the fluorescent signal, resulting in a measured increase in the fluorescent signal intensity.

The invention therefore provides binding assays that are simple and straightforward to perform. In particular, the fluorescent proximity assays of this invention simply involve contacting a sample to a particle (preferably a gold or other metallic particle) that has a molecular probe bound or otherwise attached to its surface. The molecular probe may be, for example, an antibody molecule that specifically binds to a particular protein or antigen, or the molecular probe may be a nucleic acid molecule (e.g., an oligonucleotide probe) that specifically hybridizes to a complementary nucleic acid sequence. More generally, the molecular probe may comprise any probe or molecule that specifically binds to a “target molecule” to be detected in the sample.

In preferred embodiments, molecules in the sample are directly labeled, e.g., with a fluorescent label. However, the sample molecules may also be indirectly labeled. For example, in alternative embodiments a sample may comprise unlabeled molecules (such as unlabeled nucleic acid molecule) that bind to a fluorescently tagged molecule, such as a cognate polynucleotide. The unlabeled sample molecule may bind to the fluorescent tag before or after binding to the probe molecule(s). Indeed, the fluorescent proximity assays of this invention also encompass assays that involve multiple fluorescent tags or labels, preferably with each label generating a distinct fluorescent signal.

The derivatized beads (i.e., beads having a molecular probe attached or bound to their surface) are contacted to the sample molecules under conditions such that a particular “target molecule,” if present in the sample, can bind or hybridize to the molecular probe. Binding of the target molecule to the molecular probe is then simply detected by measuring the signal from the fluorescent label. In particular, an increase in the fluorescent signal indicates that the target molecule has bound to the molecular probe and is therefore present in the sample. In alternative embodiments, a plurality of unlabeled molecules may be contacted to the derivatized beads after contacting the beads with the labeled sample molecules. In these alternative embodiments, the unlabeled target molecules may be expected to compete with labeled target molecules in the sample for binding to the molecular probe. Accordingly, the presence of target molecules in the sample can be indicated by a decrease in the fluorescent signal.

The fluorescent proximity assays of this invention are simple and straight forward to perform, and offer particular advantages compared to other assays commonly used by persons skilled in the relevant art(s). For example, the molecular probes used in these assays may be attached or bound to a nanoscale or microscale bead, and need not be attached or bound to a solid surface or substrate. It is not necessary, therefore, to contact a sample to probes that have been immobilized, e.g., in a microarray, on the surface of a glass slide or plate, to the bottom of a microtiter well, or to a membrane, as one must do for traditional “solid-phase” or “multi-phase” binding assays that are commonly used. Instead, a fluorescent proximity assay of this invention can be performed in a single, homogeneous phase where the derivatized particles are suspended in a liquid medium, such as an aqueous solution or buffer.

In addition, when practicing the fluorescent proximity assays of this invention it is not necessary to remove unbound, labeled molecules (e.g., in a washing step) before detecting binding of a target molecule to the molecular probe. Instead, binding of the probe to a target molecule may be detected by directly measuring an increase in a signal that occurs when the target molecule binds to the molecular probe. All a user needs to do is contact a sample of labeled molecules to a suspension of the derivatized beads, and measure the sample's fluorescence intensity. If the sample's fluorescence intensity increases when contacted to the derivatized beads, then a user will appreciate that the target molecule is present in the sample and has bound to an appropriate molecular probe on the beads' surface.

### 5. BRIEF DESCRIPTION OF THE DRAWINGS

FIG. 1 provides a schematic illustration of a sample comprising biotin molecules that are covalently labeled with the fluorescent label Fluorescein.

FIG. 2 illustrates an exemplary embodiment of a fluroescent proximity assay where a gold bead is derivatized with molecules of streptavidin that, in turn, specifically bind to fluorescently labeled biotin molecules in a sample. Such binding effectively brings the fluorescent label in close proximity to the gold bead.

FIG. 3 illustrates a second exemplary embodiment used to demonstrate fluorescence proximity assays of the invention. Colloidal gold is derivatized with streptavidin that, in turn, specifically binds to biotin molecules in a sample. When such beads are contacted to a sample containing both a fixed concentration of fluorescently labeled biotin and excess unlabeled biotin, the streptavidin is saturated by binding to unlabeled biotin molecules. Labeled biotin molecules are unable to bind to streptavidin on the beads' surface and remain in the bulk solution. Consequently, the fluorescent label is not held in close proximity to the gold bead.

FIGS. 4A-B schematically illustrate two, exemplary fluorescence proximity assay experiments demonstrating the present invention. In FIG. 4A, a sample containing fluorescently labeled biotin and excess unlabeled biotin is contacted to a suspension of colloidal gold beads that have streptavidin molecules attached to their surface. The streptavidin binding sites are saturated by binding to the unlabeled biotin molecules (see, FIG. 3) and no increase in the fluorescent signal is detected. In FIG. 4B a sample containing an equal concentration of fluorescently labeled biotin is contacted to the derivatized beads, without any unlabeled biotin. The fluorescenty labeled biotin molecules bind to streptavidin on the bead's surface (see, FIG. 2), and an increased fluorescent signal is observed.

FIGS. 5A-B illustrate a non-limiting model that explains one mechanism by which fluorescent signal intensity may be increased when a label is brought in close proximity to a gold or other reflective bead. FIG. 5A illustrates the exemplary situation where fluorescently labeled biotin binds to streptavidin immobilized on the surface of a gold bead. FIG. 5B illustrates the exemplary situation where streptavidin sites on a derivatized gold bead are saturated by excess unlabeled biotin molecules.

FIG. 6 provides a plot demonstrating the affect of increasing the concentration of fluorescently labeled biotin (FITC-Biotin) on observed fluorescent signal in the presence of a fixed concentration of streptavidin derivatized colloidal gold with and without excess unlabeled biotin (+Excess Biotin and −Excess Biotin, respectively).

FIG. 7 presents data from experiments where the level of a fluorescent signal was measured as a function of the concentration of streptavidin derivatized colloidal gold in the presence of a fixed concentration of fluorescently labeled biotin, and with or without excess unlabeled biotin (+Excess Biotin and −Excess Biotin, respectively).

FIG. 8 plots data from competition experiments in which colloidal gold beads having streptavidin molecules on their surface are incubated for 10 minutes in the concentration of unlabeled biotin indicated in the horizontal axis. A fixed concentration of fluorescently labeled biotin was then added to the sample, and the level of a fluorescent signal measured. Data from two repetitions of the experiment are plotted in the graph.

### 6. DETAILED DESCRIPTION OF THE INVENTION

6.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them. In addition, it is also noted that, within the context of this invention there may be employed conventional techniques of molecular biology, microbiology and recombinant DNA. Such techniques are well within the ordinary skill in the relevant art(s) and are fully explained in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al (eds.), Current Protocols in Molecular Biology, John Wiley & Sons, Inc. (1994).

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., for the presence of a particular polypeptide or nucleic acid. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In preferred embodiments samples are obtained from bone marrow.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

The terms “target” and “target molecule”, as used herein, refer to any molecule that a user may want to detect in a sample. For example, a user may want to determine whether a particular target molecule is or is not present in a sample, and/or may want to determine the molecule's abundance (i.e., the amount of that type of molecule) in the sample. The sample may be of any type and from any source. In addition, the sample may be one that is pure (e.g., contains only the target molecule) or it may contain a plurality of different molecules in addition to the target. In addition, a sample may comprise a plurality of different target molecule. That is, a sample may contain a plurality of different types of molecules, each of which a user may wish to detect. Exemplary target molecules include nucleic acid molecules that have a particular nucleotide sequence (e.g., RNA or DNA molecules corresponding to a particular genetic transcript) and polypeptide molecules that have a particular amino acid sequence (e.g., molecules of a particular protein).

The terms “probe” and “molecular probe” refer to any molecule that specifically binds to a target molecule. Molecular probes may therefore be used to detect target molecules, e.g., in a specific binding assay. Preferred, exemplary, molecular probes include nucleic acid molecules (e.g., oligonucleotides) that specifically hybridize to a complementary target nucleic acid sequence, and antibodies that specifically bind to a target polypeptide or target antigen.

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells.

Typically, a ligand is a soluble molecule and the receptor is attached or otherwise immobilized on a surface or a substrate. For example, a receptor may be an integral membrane protein (i.e., a protein expressed on the surface of a cell). As used to described the present invention, a ligand may also be a particular target molecule in a sample (for example a nucleic acid or a polypeptide of interest), and a receptor may be a molecular probe that specifically binds to the target.

6.2. Fluorescence Proximity Assays

The present invention may be readily understood in terms of exemplary embodiments that are illustrated in the accompanying figures and described here below. However, the use of these or other examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

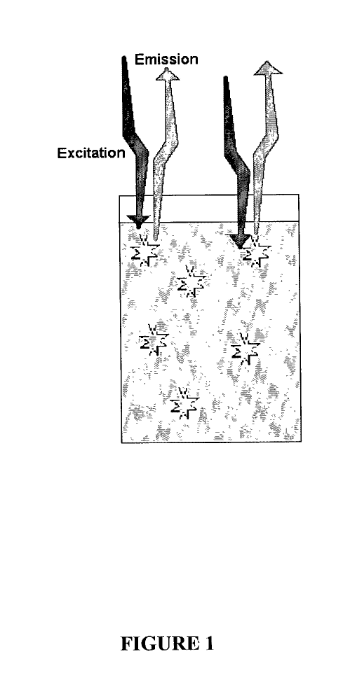

FIG. 1 schematically illustrates a solution of sample molecules that are labeled with a detectable label. In the exemplary embodiment depicted by FIG. 1, the sample comprises streptavidin molecules that are covalently labeled with the fluorescent label Fluorescein. However, the sample may be a sample of any type of molecules and may be from any source. In preferred embodiments the sample is a biological sample, such as a sample of proteins and/or nucleic acids that may be derived from a cell or other biological source. Such samples can be readily obtained or provided using conventional techniques that are well known, e.g., in the arts of molecular biology, microbiology, and recombinant DNA technology. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

Preferably, the detectable label is a fluorescent label. Such labels generally emit a detectable signal of fluorescent light when irradiated with light having a particular energy or wavelength, referred to as the “excitation light” or the “excitation energy.” Generally, each different fluorescent label will emit fluorescent light having a particular wavelength or wavelengths; i.e., the label is said to have a particular “emission spectrum” that is preferably characteristic of the label. While preferred fluorescent labels generally absorb and emit light at visible wavelengths, fluorescent labels that either absorb or emit light with shorter or longer wavelengths than visible light (i.e., ultra-violet or infrared light) may also be used.

A variety of fluorescent labels are well known in the art, which can be used in the methods of this invention. Exemplary fluorescent labels include fluorescein, lissamine, phycoerythrin, rhodamine (Perkin Elmer Cetus), FluorX (Amersham), Cy2, Cy3, Cy3.5, Cy5, Cy5.5 and Cy7. Still others have also been described in the literature. See, for example, Kricka, Nonisotopic DNA Probe Techniques 1992, Academic Press, San Diego, Calif.

Molecules in a sample may be either directly or indirectly labeled. Generally, molecules that are directly labeled are directly bound to a detectable label (e.g., a fluorescent molecule) for example by covalent or non-covalent bonding. By contrast, molecules in a sample that has been indirectly labeled do not have the label bound directly to them. Instead labeled “conjugate” molecules are added that specifically bind to a target molecule in the sample, and have a detectable label bound to them. Thus, target molecules in a sample are effectively labeled by binding to a conjugate molecule that is, itself, detectably labeled. Indirect labeling methods are particularly preferred in embodiments where two or more different target molecules are detected in a fluorescence proximity assay. In such embodiments, two or more detectable labels may be used that are distinguishable from each other, e.g., by having distinct emission spectra.

As a particular example, in embodiments where one or more different proteins or antigens are detected in a fluorescence proximity assay, a target protein or antigen may be indirectly labeled by binding to an antibody, which specifically binds to the target protein and is detectably labeled. The target protein may bind to probe molecules before or after binding to the labeled antibody. In such embodiments, a plurality of different proteins or antigens may be simultaneously detected by simply adding a plurality of different labeled antibodies to the sample, in which each different antibody binds to a particular target protein and is labeled with a different label. The sample may then be contacted to beads that have a plurality of different probe molecules (e.g., an antibody specific for each target protein) attached to their surface. The presence of each target protein may then be detected by simply detecting an increase in the fluorescence signal for each different label. In such embodiments, the number of different protein or antigen molecules that may be detected will generally be limited only by the number of labels having emission spectra that may be separately distinguished from each other. Also, in such embodiments both the labeled antibody molecules and the antibody probe molecules are preferably selected so that binding of a labeled antibody to a particular protein does not significantly affect that protein's binding to its respective antibody probe, and vice versa.

Similar embodiments are also provided where a target nucleic acid molecule may be indirectly labeled with a polynucleotide (e.g., an oligonucleotide molecule) having a sequence that is complementary to a sequence in the target nucleic acid and/or specifically hybridizes thereto. The complementary nucleic acid is preferably, in turn, labeled with a detectable label. In such embodiments, the target nucleic acid molecule also binds to a molecular probe, which is preferably a second polynucleotide (e.g., a second oligonucleotide molecule) having a sequence that is complementary to another sequence in the target nucleic acid and/or specifically hybridizes thereto (preferably, without affecting hybridization of the labeled polynucleotide). As a skilled artisan will readily appreciate, such embodiments may be readily to adapted to assays where a plurality of different target nucleic acid molecules are simultaneously detected, e.g., by indirectly labeling each target nucleic acid with a distinct label. Such embodiments are similar to the embodiments described, above, for detecting different proteins.

In still other embodiments, a target molecule may be labeled with a fluorescence emitter moiety and also with either a fluorescence enhancer moiety, a fluorescence quencher moiety, or both. The use of a fluorescence enhancer or quencher moiety is useful, e.g., in embodiments of the invention that combine methods of fluorescence resonance energy transfer (FRET) in a fluorescence proximity assay. See, U.S. Pat. Nos. 5,573,906 and 6,090,552 for descriptions of exemplary binding assays that use FRET to enhance a fluorescence signal indicating binding. As an example, a fluorescence enhancer moiety may be used to further enhance a fluorescent signal when the target molecule binds to a molecular probe. Alternatively, a quencher moiety may be used to “quench” or suppress a signal from a fluorescent label when the target molecule is not bound to a probe. Such embodiments are useful, therefore, to improve the increased fluorescence that indicates binding in a fluorescence proximity assay.

As a particular example and not by way of limitation, a fluorescence enhancer moiety may be associated with a particle or bead used in the present invention or, alternatively, with a molecular probe that is in turn associated (e.g., attached to) such a particle or bead. Consequently, binding of the molecular probe to a fluorescently labeled target molecule will preferably bring the fluorescence enhancer into sufficient proximity with a fluorescent label of the target molecule, so that the detectable signal from that label is enhanced or increased. Because the particle or bead used in the present invention further increases the intensity of a fluorescent signal, such an assay offers further improvements in signal enhancement beyond existing FRET assays that are known in the art.

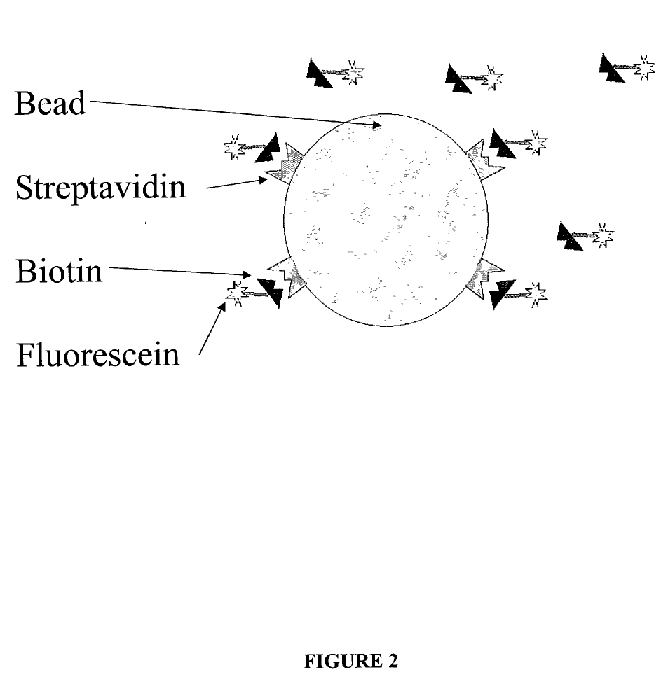

According to the fluorescence proximity assay methods for this invention, molecular probes that specifically bind or hybridize to a particular target molecule may be bound or attached to the surface of a particle or bead, as illustrated schematically in FIG. 2. Preferably, the particle or bead is made of gold or other metal. However, the bead or particle may be composed of any material capable of reflecting light or energy emitted, e.g., from a fluorescent label. The bead or particle may be made entirely of the reflective material, or it may simply be “coated” with the material so as to have a reflective surface (e.g., a gold coated bead or particle). Any colloidal metallic material, such as colloidal silver or aluminum, may be used in these methods (see, Enderlein Biophys J. 2000, 78:2151-2158 for other examplary materials which may be used). In preferred embodiments the material is colloidal gold.

The particles and beads are preferably small enough that the particle can be suspended, e.g., in a homoegenous colloid. Thus, colloidal particles (e.g., colloidal gold) are particularly preferred. Such particles typically have an average diameter that is between about 1 nm and a few hundred micrometers. In preferred embodiments, average particle sizes are between about 1.4 nm and 100 nm. More preferably, the particle diameters are (on average) no more than about 10 nm in diameter, with an average particle diameter of 10 nm being particularly preferred.

The molecular probe may be any type of molecule or probe that is capable of specifically recognizing and/or binding to a target molecule of interest to a user. For instance, in the exemplary embodiment illustrated in FIG. 2 the molecular probe comprises molecules of streptavidin that specifically bind to biotin molecules in a sample. However, in more preferred embodiments the molecular probe may be, e.g., an antibody molecule that specifically binds to a particular protein or antigen of interest or, alternatively, a nucleic acid molecule (e.g., an oligonucleotide probe) that specifically hybridizes to a complementary sequence in a target nucleic acid (for example, a genetic transcript) of interest.

The molecular probes may be readily attached or immobilized to a bead or particle using conventional techniques that are already known in the art and, in many instances, are commercially available. As an example, and not be way of limitation, particles or beads may be coated with streptavidin which, in turn, may bind to biotinylated molecular probe molecules. Alternatively, a particle or bead may be coated with either protein A or protein G for antibody capture. Techniques are also known and available for coating particles of colloidal gold with amine groups. Such groups may be chemically modified, allowing them to covalently bind to ligands, e.g., at free amine or thiol groups. Alternatively, a bead or particle used in the fluorescence proximity assays of this invention may be coated with polylysine for immobilizing polynucleotide probes. Chemistries for immobilizing carbohydrate molecules are also known in the art and may be used in these methods.

The beads or particles used in a fluorescence proximity assay may also be labeled, preferably with a different label that is distinguishable from the label(s) used for the target molecule(s) in a sample. For example, a colloidal bead may be derivatized with a fluorescent label in addition to a molecular probe. The fluorescence signal from that label may then be used, e.g., to visualize and/or quantitate the number of beads within a sample. This information may then be used to normalize the second fluorescent signal (i.e., from the sample) which is used to indicate binding of the target molecule(s). In such embodiments, the beads or particles may be directly labeled, e.g., by directly binding a fluorophore to the bead's surface. Alternatively, the beads or particles may be indirectly labeled. For example, in certain preferred embodiments a label may be bound (either directly or indirectly) to the molecular probe which, in turn, is bound or attached to a bead or particle.

In a preferred embodiment, an assay of the present invention may be practiced in a homogeneous phase, such as in a liquid solution or colloidal suspension. In such embodiments, a liquid sample that contains or is suspected to contain one or more target molecules of interest can be simply contacted to a colloidal suspension of particles or beads having the moleculare probe(s) attached thereto. The reagents may be combined in any order. For example, target molecules in a sample may first be detectably labeled (either directly or indirectly), for instance by contacting the sample with an antibody, nucleic acid or other molecule that specifically binds to target molecules of interest and which has a detectable label attached thereto. After the sample has been detectably labeled, the sample may then be contacted to a colloidal suspension of beads that have the molecular probe(s) attached or bound thereto, under conditions such that the labeled target molecule(s) may bind to molecular probes attached to the metallic beads or particles. Alternatively, however, a sample of target molecules may first be contacted to the suspension or colloidal gold or other beads so that the target molecules bind to molecular probes on the beads, and target molecules in the sample may then be detectably labeled.

Such homogenous assays offer a great advantage over other detection assays currently in use since there is no need to separate unbound probes or beads from the sample. Instead, the target molecule(s) of interests may be readily detected by simply detecting an increase in the fluorescence signal. Generally, the increase in fluorescence intensity will be proportional to the number of labeled target molecules binding to molecular probes on the colloidal beads or particles, which is in turn related to the quantity of target molecules present in the sample. Thus, the amount of target molecules present can also be readily determined or measured in such assays, by simply measuring or determining the increase in intensity of the fluorescent signal.

In other embodiments, an assay of the invention may be practiced as a heterogeneous phase assay, e.g., to detect the binding or hybridization of molecules on a solid surface or support (e.g., on a substrate). For instance, such a fluorescence proximity assay may be readily adapted to detect the binding or hybridization of molecules to a microarray, such as an array of nucleic acids or antibodies attached to a solid surface.

As an illustration and not by way of limitation, a sample containing or suspected of containing one or more target molecules of interest may be contacted to a solid surface or support that has a first set of molecular probes attached thereto. These molecular probes are preferably molecules that specifically hybridize or bind to particular target molecules of interest and may be, for example, oligonucleotide probes that specifically hybridize to a particular nucleic acid sequence of interest (e.g., an oligonucleotide array), or antibody probes that specifically bind to a particular polypeptide or protein of interest (e.g., an antibody array). Beads or particles that have a second set of molecular probes attached thereto may then also be contacted to the solid surface or support. In particular, the molecular probes in this second set of molecular probes are preferably ones that also specifically hybridize or bind to target molecules of interest. Preferably, the molecular probes in this second set of molecular probes bind or hybridize to a domain or region of the target molecules (e.g., a particular nucleotide sequence or a particular epitope) which is different from the domain or region recognized by the first set of molecular probes. Thus, binding of the first set of molecular probes to the target molecule(s) preferably does not interfere with the binding of the second set of the molecular probes and vice versa.

As in the homogeneous phase assays described, supra, target molecules in the sample are preferably detectably labeled (either directly or indirectly), with fluorescent labels being particularly preferred. As an example and not by way of limitation, in embodiments where the target molecules are nucleic acid molecules, the sample may be a sample of labeled nucleic acids (e.g., cDNA or cRNA) prepared, e.g., by the reverse transcription of an RNA sample in the presence of fluorescently labeled nucleotide triphosphates. Alternatively, in embodiments where the target molecules are polypeptides, the sample may be a sample of fluorescent polypeptide molecules prepared, e.g., using one or more fluorescently labeled amino acid residues.

As another example, target molecules may be labeled by contacting the sample with a detectable moiety that binds non-specifically to a molecular species (e.g., nucleic acid molecules or polypeptides) that include the target molecules of interest. For instance, in embodiments where the target molecules are nucleic acid molecules, the target molecules may be labeled by contacting the sample with an intercalating dye such as SYBR Green, TO, TO6, Propidium2, AID3, eithidium bromide, YOYO or an acridine dye. In still another embodiment, the target molecules may be indirectly labeled by labeling the first set of molecular probes (directly or indirectly) which are attached to the solid surface or support. In such embodiments, binding of the target molecules to the first set of molecular probes can serve a dual function of (i) anchoring or attaching the target molecules of interest to the solid surface or substrate, and (ii) indirectly labeling the target molecules of interest.

As in the homogenous assay format, target molecules of interest may be readily detected by simply detecting the increase in fluorescent signal intensity that occurs upon binding of the target molecule(s) to molecular probes attached to the beads or particle. Accordingly, the assay offer an advantage over existing heterologous phase detection assays in that it eliminates the need to perform an additional “washing” step to remove unbound molecules or label.

Those skilled in the art will appreciate that in such heterologous formats, different target molecules may be simultaneously detected and distinguished in a single assay without the need for differential labeling. For instance, such formats are particularly well suited for use with “addressable” arrays in which each molecular probe in the first set of molecular probes is attached at a unique, known location (i.e., at a known “address”) on the solid surface or support. Thus the identity of each target molecule detected in such an assay may be readily determined from the position or “address” of the detected increase in fluorescence intensity on the surface.

The invention also provides kits, which a user may conveniently use to perform a fluorescence proximity assay of the invention. Such kits, which are considered part of the invention, contain materials and reagents that are conveniently packaged for performing a fluorescence proximity assay of the invention, and preferably also contain instructions for the kit's use.

For example, preferred kits of the invention may contain a collection of beads or particles, e.g., in colloidal suspension, that may be used in a fluorescence proximity assay. The beads or particles may be derivatized with a molecular probe, or with a plurality of different molecular probes. Alternatively, the kit may contain instructions for a user to derivatize the particles with an appropriate molecular probe or probes. In such alternative embodiments, the molecular probe or probes may be packaged separately in the kit, or they may be provided separately, e.g., by a user. The kits of the invention may also contain additional reagents that can be used, e.g., to prepare or label a sample of molecules for the fluorescence proximity assay. For instance, in embodiments where a sample is indirectly labeled, a kit of the invention may contain one or more additional, labeled probes that specifically bind to one or more particular target molecule (e.g., at the same time the target molecules are bound to a molecular probe on the surface of a particle or bead).

### 7. EXAMPLE

The invention is further described here by means of the following example, In particular, this example describes the implementation of one exemplary embodiment of a fluorescence proximity assay of the invention and presents data demonstrating that assay's affectivity. The example is provided merely to clarify the description of the invention, and the invention is not limited to any particular embodiment described or demonstrated herein.

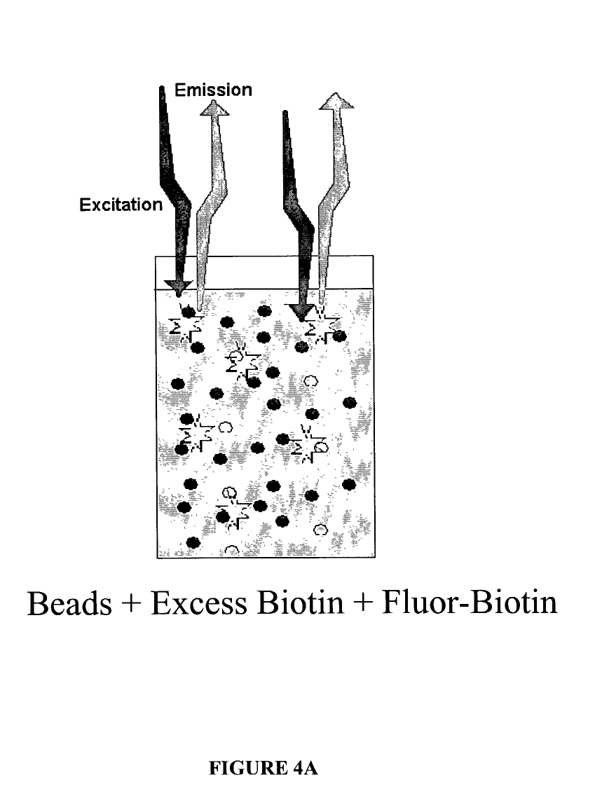

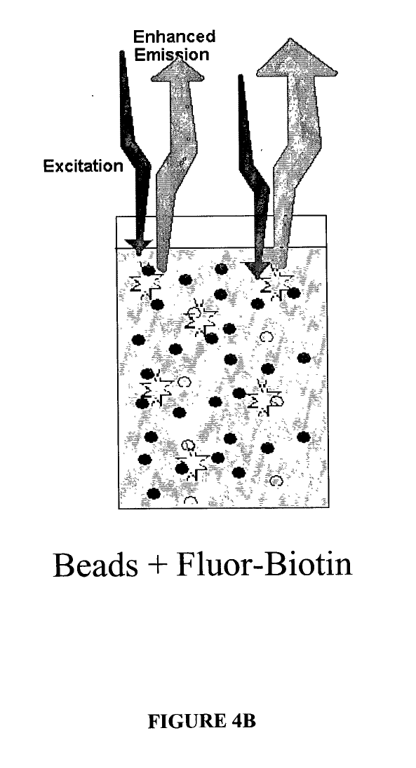

Applicants have found that, surprisingly, fluorescent excitation and emission wavelengths (e.g., from a fluorescently labeled target molecule) are not quenched or absorbed by close proximity to a gold or other reflective surface (e.g., by binding to a molecular probe immobilized on the surface of a gold bead). Indeed, such emissions are actually increased. These finding are illustrated schematically in FIGS. 4A and 4B.

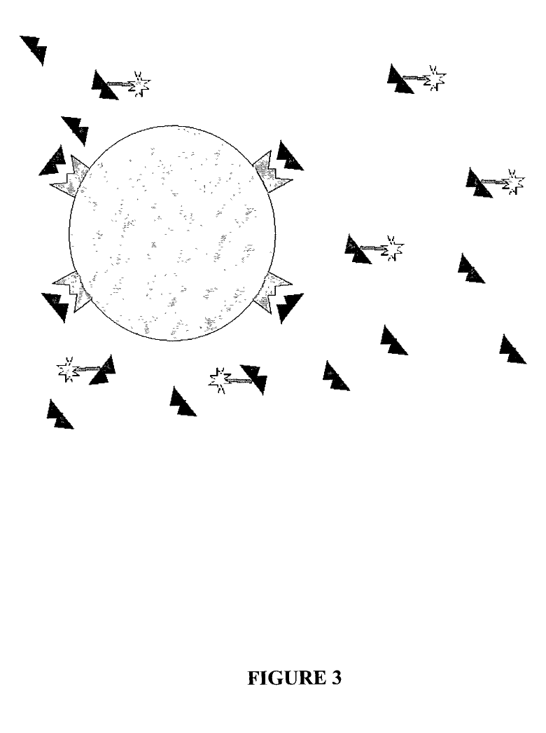

FIG. 4A illustrates one example where streptavidin coated particles of colloidal gold (10 nm average diameter) are added to a sample that contains both labeled (with fluorescein) and unlabeled molecules of biotin. However, the unlabeled biotin molecules are present in excess (i.e., at greater concentration than labeled biotin). Consequently, the unlabeled biotin molecules successfully out compete the labeled biotin molecules for binding to the beads' surface, as illustrated in FIG. 3. The fluorescently labeled biotin molecules remain unbound, in the solution phase and, as a result, the fluorescent signal detected in this sample does not increase when the particles of colloidal gold are added.

In contrast, the situation illustrated in FIG. 4B is one where the sample contains the same concentration of fluorescently labeled biotin as in FIG. 4A, but contains no unlabeled biotin. As a result, the fluorescently labeled molecules bind to streptavidin immobilized on the surface of the gold beads (FIG. 2), thereby bringing the fluorescent label in close proximity to the gold particles. Surprisingly, the level of fluorescent signal observed in this situation has actually increased, compared to the fluorescent signal in FIG. 4A. Thus, binding of the labeled target molecules (in this particular example, biotin) to the molecular probe (in this particular example, streptavidin) is readily detected by simply detecting the increase of the fluorescent signal.

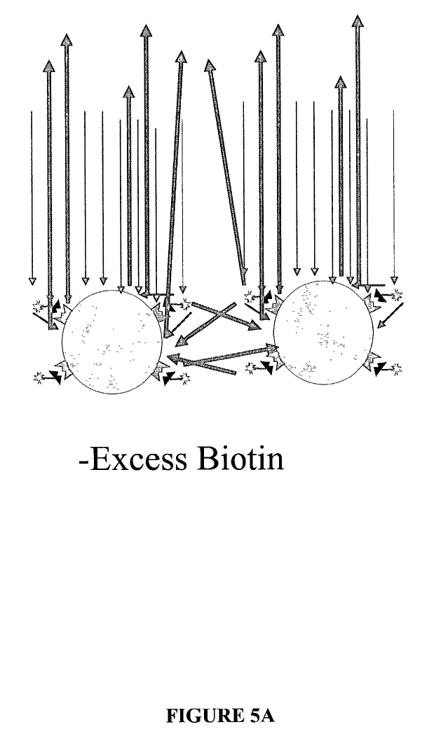

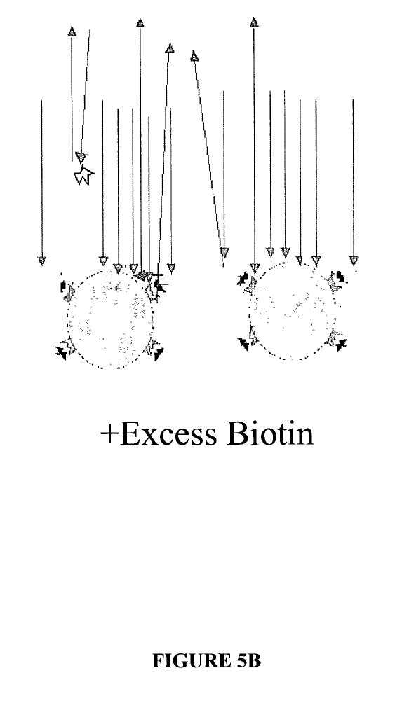

Without being limited to any particular theory or mechanism of action, the observed increase in fluorescence intensity is believed to be due, at least in part, to reflection of emitted light by the gold beads. This model is schematically illustrated in FIGS. 5A and 5B. Briefly, in experiments where unlabeled biotin molecules saturate streptavidin binding, labeled biotin molecules are in free solution. Excited light from the fluorophore ie emitted in all directions and light that is emitted away from the detector is “lost” (FIG. 5B). FIG. 5A illustrates the experiment where excess unlabeled biotin is removed, and fluorescently labeled biotin molecules bind to the gold beads. Again, fluorescent light is emitted in all directions. However, because the label is bound in tight proximity to a gold bead, light emitted towards the bead is reflected back, towards the solution. Similarly, excitation light (i.e., light or other energy used to stimulate fluorescence) may also be reflected by the gold beads, increasing the probability that the light will stimulate a fluorophore held in close proximity to a bead's surface. Hence, fluorescence proximity assays of the invention are preferably implemented with gold or gold coated beads. However, any bead having a surface capable of reflecting fluorescent light (i.e., light emitted by a fluorophore) or excitation light (i.e., light or other energy used to excite a fluorophore) may be used. Particular examples, other colloidal metals may also be used in these methods include colloidal silver or aluminum. See, also, the materials used by Enderlein (Biophys. J. 2000, 78:2151-2158).

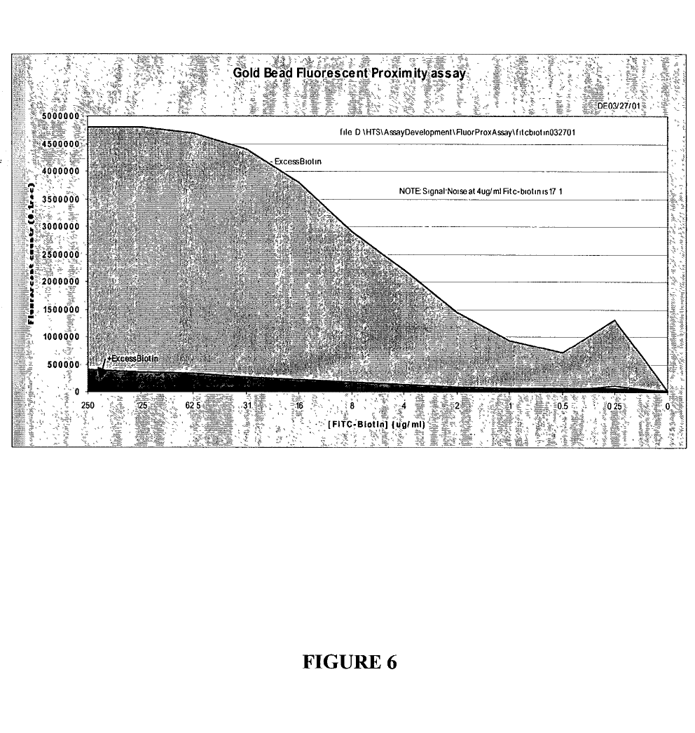

Quantitative results from the above-described experiments are presented in FIGS. 6-8. In particular, FIG. 6 shows the effect of increasing the concentration of fluorescently labeled biotin (FITC-Biotin) on the observed fluorescent signal in the presence of a fixed concentration of streptavidin derivatized colloidal gold (10 nm average diameter).

For these experiments, a stock suspension of streptavidin derivatized colloidal gold (10 nm average particle diameter) was obtained from Sigma Aldrich (St. Louis, Mo.). The gold particles were suspended in 10 mM phosphate buffer with 1% bovine serum albumin (BSA) and 20% glycerol. The suspension's absorbance of 520 nm light (A₅₂₀) was measured and recorded as 2.5. 4 μl of the stock colloidal gold suspension was added to each well of a 96-well microtiter plate. A measured volume of fluorescently labeled biotin (FITC-biotin) was also added to each the microtiter wells, which were then brought up to a final volume of 50 μl. In a set of control experiments, 10 μl of unlabeled biotin (10 mg/ml) solution was also added to each wells before final dilution to 50 μl.

FIG. 6 indicates the measured fluorescence activity as a function of the FITC-biotin concentration within the different wells. When streptavidin binding is saturated in the control experiments by the excess unlabeled biotin in the samples, the observed fluorescent signal is simply proportional to the FITC-biotin concentration, as expected. This data is shown in the bottom portion of the graph set forth in FIG. 6 (+Excess Biotin). By contrast, when the excess unlabeled biotin is removed (−Excess Biotin) the observed fluorescence intensities increase by as much as 10-fold, even though the total concentration of the fluorescent label is the same as in the control experiments.

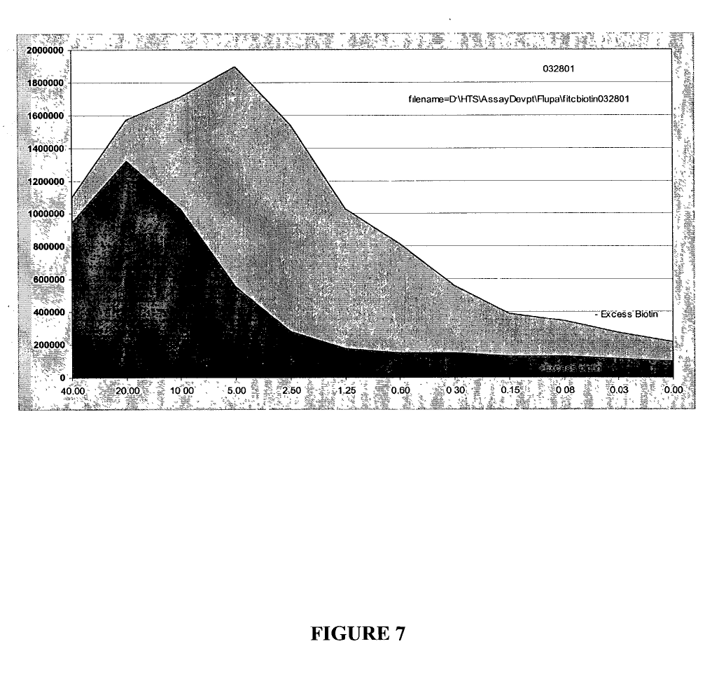

FIG. 7 shows data from similar experiments in which the concentration of gold beads was varied for a fixed concentration of fluorescently labeled target molecules. More specifically, varied volumes (indicated on the horizontal axis in FIG. 7) of the stock colloidal gold suspension were added to wells of a microtiter plate and diluted to a total volume of 40 μl. To these volumes, 10 μl of a stock FITC-biotin (4.0 μg/ml) solution was also added and, for control experiments, 10 μl of unlabeled biotin (1 mg/ml). Fluorescence signals measured for the samples in the presence of excess, unlabeled biotin (+Excess Biotin) and without the unlabeled biotin (−Excess Biotin) are plotted in FIG. 7. For any given concentration of gold beads, there is still a decrease in the fluorescent signal observed when unlabeled biotin is added to the sample. The difference is most pronounced when about 2.5 μl of the stock colloidal gold suspension is diluted to 50 μl. Here, binding of labeled biotin to the beads enhances the signal-to-noise ratio of the fluorescence intensity by about 10-fold (i.e., the signal-to-noise ratio is approximately 10 to 1).

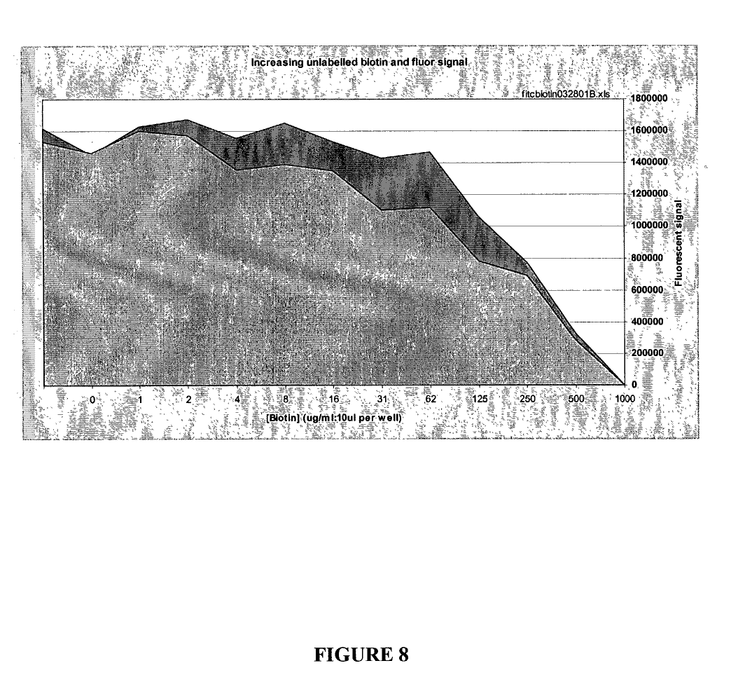

Additional experiments were performed to verify that the labeled and unlabeled biotin molecules are actually competing for the same binding site on the derivatized gold beads, and data from those experiments is presented in FIG. 8. Specifically, about 0.45 μl of the stock colloidal gold suspension was added to each well of a microtiter plate. Serial dilutions of FITC-biotin were also prepared having the final concentrations of unlabeled biotin indicated along the horizontal axis in FIG. 8, and 10 μl of each dilution was added to a microtiter well with the colloidal gold. The suspensions were incubated for 10 minutes, followed by the addition of 10 μl of FITC-biotin (4 μg/ml) to each well. The fluorescent signal from each well was then detected and measured, and these measured values are plotted in FIG. 8 as a function of the unlabeled biotin concentration. The unlabeled biotin effectively decreased the fluorescent signal in a dose dependent manner. To verify these results, the experiment was repeated a second time, and the results from each experiment are separately plotted in FIG. 8.

8. References Cited

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited or discussed in this specification are incorporated herein by reference in their entirety and to the same extent as if each reference was individually incorporated by reference.

What is claimed is:

1. A method for detecting a target molecule in a sample, which method comprises:
(a) contacting the sample with a particle having a molecular probe attached thereto,
which molecular probe is capable of specifically binding to a target molecule in the sample, and
which particle increases a signal from a detectable label when the target molecule is bound to the molecular probe; and
(b) detecting an increase in the signal from the detectable label,
wherein an increase in the signal indicates that the target molecule is present in the sample.

2. A method according to claim 1 in which the particle comprises a surface capable of reflecting a signal from the detectable label.

3. A method according to claim 2 in which the surface comprises a colloidal metallic material.

4. A method according to claim 3 in which the colloidal metallic material is selected from the group consisting of gold, siliver and aluminum.

5. A method according to claim 1 in which the particle comprises colloidal gold.

6. A method according to claim 1 in which the particle has a diameter between about 1.4 and 100 nm.

7. A method according to claim 1 in which the particle has a diameter less than or equal to about 10 nm.

8. A method according to claim 1 in which the target molecular is a nucleic acid molecule.

9. A method according to claim 8 wherein the molecular probe is a second nucleic acid molecule capable of specifically hybridizing to the target nucleic acid molecule.

10. A method according to claim 1 in which the target molecule is a polypeptide molecule.

11. A method according to claim 10 in which the molecular probe is an antibody capable of specifically binding to the target polypeptide molecule.

12. A method according to claim 1 wherein the detectable label is a fluorescent label.

13. A method according to claim 12 in which the fluorescent label is lissamine, phycoerythrin, rhodamine, FluorX or a cyanimin dye.

14. A method according to claim 12 in which the detectable label is an intercalating dye.

15. A method according to claim 14 in which the intercalating dye is selected from the group consisting of SYBR Green, TO, TO6, Propidium iodide 2, Propidium iodide 3, YOYO and ethidium bromide.

16. A method according to claim 1 in which the detectable label is bound to target molecules in the sample

17. A method according to claim 16 in which the detectable label is bound to a conjugate molecule, and said conjugate molecule binds to target molecules in the sample when added thereto.

18. A method according to claim 1,
in which the detectable label is a fluorescent label, and
wherein a fluorescence enhancer moiety is also associated
(i) with the particle or
(ii) with the molecular probe associated with said particle
such that the signal from the fluorescent label is enhanced by said enhancer moiety upon binding of the target molecular to the molecular probe.

19. A method according to claim 1 which method is conducted in a homogeneous phase.

20. A method according to claim 19 in which the target molecule specifically binds to the molecular probe is a colloidal suspension of particles.

21. A method according to claim 1 in which the target molecule is attached to a solid surface or support.

22. A method for detecting a target molecule in a sample, which method comprises,
contacting the sample to a surface or support having a first molecular probe associated therewith, said first molecular probe being capable of specifically binding to a target molecule in the sample;
(b) contacting to the surface or support a particle having a second molecular probe associated therewith,
said second molecular probe being capable of specifically binding to the target molecule when said target molecule is bound to the first molecular probe on the surface or support, and
wherein the particle increases a signal from a detectable label when the target molecule is bound to the second molecular probe; and
(c) detecting an increase in the signal from the detectable label,
wherein an increase in the signal indicates that the target molecule is present in the sample.

23. A method according to claim 22 in which the detectable label is associated with the first molecular probe on the solid surface or support.

24. A method according to claim 22 in which the detectable label is associated with the target molecule.

25. A method according to claim 22 in which the particle comprises colloidal gold.

26. A method according to claim 22 in which the detectable label is a fluorescent label.

27. A method according to claim 22 in which the target molecule is a nucleic acid molecule.

28. A method according to claim 22 in which the target molecule is a polypeptide molecule.

29. A method according to claim 22 in which the solid surface or support comprises a plurality of molecular probes, each different molecular probe being capable of specifically binding to a different target molecule in the sample.
